(19) United States
(12) Patent Application Publication
(10) Pub. No.: US 2017/0190107 A1
(43) Pub. Date: Jul. 6, 2017

(54) MANUFACTURING APPARATUS FOR USE IN LOW-TEMPERATURE HIGH-SPEED MANUFACTURING OF SUPPORT STRUCTURE AND MANUFACTURING METHOD FOR USE IN LOW-TEMPERATURE HIGH-SPEED MANUFACTURING OF SUPPORT STRUCTURE

(71) Applicant: NATIONAL CENTRAL UNIVERSITY, Taoyuan City (TW)

(72) Inventors: CHAO-YAUG LIAO, TAIPEI CITY (TW); CHING-SHIOW TSENG, TAOYUAN CITY (TW); FANG-CHIEH TU, TAOYUAN CITY (TW); YEN-SHENG LIN, TAICHUNG CITY (TW); WEI-JEN WU, TAOYUAN CITY (TW)

(21) Appl. No.: 15/171,157

(22) Filed: Jun. 2, 2016

(30) Foreign Application Priority Data
Jan. 4, 2016 (TW) 105100100

(51) Int. Cl. B29C 67/00; B33Y 10/00; A61F 2/02; B33Y 80/00; B33Y 70/00; B29C 35/16; B33Y 30/00

(52) U.S. Cl. CPC B29C 67/0055; B29C 67/0092; B29C 35/16; B33Y 10/00; B33Y 30/00; B33Y 80/00; B33Y 70/00; A61F 2/02; B29C 2035/1625; B29K 2105/0058

(57) ABSTRACT

A manufacturing apparatus for use in the low-temperature high-speed manufacturing of a support structure is introduced. The support structure thus manufactured is for supporting a low-temperature manufacturing scaffold for use in tissue engineering. The manufacturing apparatus includes a frame; a ring-shaped thermally conductive member disposed on the frame; a thermally conductive platform disposed centrally at the ring-shaped thermally conductive member and having an edge in direct contact with an inner wall of the ring-shaped thermally conductive member, wherein a space is defined by and between the thermally conductive platform and the ring-shaped thermally conductive member; a low temperature generating mechanism connected to the ring-shaped thermally conductive member and the thermally conductive platform to cool down the ring-shaped thermally conductive member and the thermally conductive platform; and a spray member disposed above the thermally conductive platform to spray a support material into the space.

### CROSS-REFERENCE TO RELATED APPLICATION

This non-provisional application claims priority under 35 U.S.C. §119(a) on Patent Application No(s). 105100100 filed in Taiwan, R.O.C. on Jan. 4, 2016, the entire contents of which are hereby incorporated by reference.

### FIELD OF THE INVENTION

The present invention relates to manufacturing apparatuses and, more particularly, to a manufacturing apparatus for use in the low-temperature high-speed manufacturing of a support structure for supporting a low-temperature manufacturing scaffold for use in tissue engineering.

### BACKGROUND OF THE INVENTION

A conventional low-temperature manufacturing scaffold for use in tissue engineering is manufactured by depositing a scaffold material layer, wherein the upper layer structure of the scaffold thus manufactured must be fully deposited on its lower layer structure in order to finalize the shape of the scaffold thus manufactured. If the low-temperature manufacturing scaffold for use in tissue engineering has a specific structure, such as a suspended end, the scaffold cannot be directly manufactured but requires an additional support structure (see Ming C. Leu and Diego A. Garcia. Development of freeze-form extrusion fabrication with use of sacrificial material, Journal of Manufacturing Science and Engineering December 2014, Vol. 136/061014-1-9.)

A conventional support structure is made of a support material which is different from that the conventional low-temperature manufacturing scaffold for use in tissue engineering is made of. The low-temperature manufacturing of a support structure, which is effectuated in a way similar to the manufacturing of the low-temperature manufacturing scaffold for use in tissue engineering, entails squeezing out a support material with a nozzle and then depositing it to therefore form a support structure. The aforesaid manufacturing process is not only slow but also unfavorable to the deposition of the scaffold material layer of the low-temperature manufacturing scaffold for use in tissue engineering (see Frances D. Bryant and Ming C. Leu. Modeling and experimental results of concentration with support material in rapid freeze prototyping, Rapid Prototyping Journal 2009, Vol. 15 Iss5 pp. 317-324.)

The conventional support structure supports, from below, the scaffold material layer of the low-temperature manufacturing scaffold for use in tissue engineering. If the scaffold material layer of the low-temperature manufacturing scaffold for use in tissue engineering is misaligned during the deposition process and thus goes beyond the support capacity of a support structure, the scaffold material cannot be supported by the support structure.

Accordingly, it is imperative to provide a manufacturing apparatus for use in the low-temperature high-speed manufacturing of a support structure to effectuate the low-temperature high-speed manufacturing of a support structure, facilitate the deposition of a scaffold material layer of a low-temperature manufacturing scaffold for use in tissue engineering on the support structure, and thus enable the low-temperature manufacturing scaffold for use in tissue engineering to form a specific structure.

### SUMMARY OF THE INVENTION

It is an objective of the present invention to provide a manufacturing apparatus for use in the low-temperature high-speed manufacturing of a support structure to effectuate the low-temperature high-speed manufacturing of a support structure, facilitate the deposition of a scaffold material layer of a low-temperature manufacturing scaffold for use in tissue engineering on the support structure, and thus enable the low-temperature manufacturing scaffold for use in tissue engineering to form a specific structure.

In order to achieve the above and other objectives, the present invention provides a manufacturing apparatus for use in the low-temperature high-speed manufacturing of a support structure, and the support structure thus manufactured is for supporting a low-temperature manufacturing scaffold for use in tissue engineering. The manufacturing apparatus comprises: a frame; a ring-shaped thermally conductive member disposed on the frame; a thermally conductive platform disposed centrally at the ring-shaped thermally conductive member and having an edge in direct contact with an inner wall of the ring-shaped thermally conductive member, wherein a space is defined by and between the thermally conductive platform and the ring-shaped thermally conductive member; a low temperature generating mechanism connected to the ring-shaped thermally conductive member and the thermally conductive platform to cool down the ring-shaped thermally conductive member and the thermally conductive platform; and a spray member disposed above the thermally conductive platform to spray a support material into the space.

Regarding the manufacturing apparatus of the present invention, the support material has a water content of 100%.

Regarding the manufacturing apparatus of the present invention, the support material comprises an alcohol solution.

Regarding the manufacturing apparatus of the present invention, the spray member sprays the support material extensively.

Regarding the manufacturing apparatus of the present invention, the spray member is fan-shaped, round or elliptical.

In order to achieve the above and other objectives, the present invention further provides a manufacturing method for use in the low-temperature high-speed manufacturing of a support structure, with the support structure supporting a low-temperature manufacturing scaffold for use in tissue engineering, the manufacturing method comprising the steps of: S100: providing the manufacturing apparatus of any one of claims 1-9; S300: injecting a scaffold material layer for use in tissue engineering into a space and then depositing the scaffold material layer on the thermally conductive platform to allow the scaffold material layer deposited on the thermally conductive platform to solidify at low temperature; S500: spraying a support material into the space with the spray member; S700: enabling the support material reaching the thermally conductive platform to be solidified at low temperature until the support material is of a less height than or same height as the scaffold material layer frozen; and S900: injecting and depositing the scaffold material layer on one of the scaffold material layer frozen and the support material frozen, thereby allowing the scaffold material layer to solidify at low temperature.

In conclusion, the present invention provides a manufacturing apparatus for use in the low-temperature high-speed manufacturing of a support structure, facilitate the deposition of a scaffold material layer of a low-temperature manufacturing scaffold for use in tissue engineering on the support structure, and thus enable the low-temperature manufacturing scaffold for use in tissue engineering to form a specific structure.

### BRIEF DESCRIPTION OF THE DRAWINGS

Objectives, features, and advantages of the present invention are hereunder illustrated with specific embodiments in conjunction with the accompanying drawings, in which:

FIG. 1 is a schematic view of a manufacturing apparatus for use in the low-temperature high-speed manufacturing of a support structure according to the present invention;

FIG. 2 is a cross-sectional view of a manufacturing apparatus according to the present invention;

FIG. 3 through FIG. 6 are cross-sectional views of a manufacturing apparatus for manufacturing the support structure according to the present invention;

FIG. 7A is a side view of a low-temperature manufacturing scaffold for use in tissue engineering according to the present invention, showing that the scaffold has a support structure;

FIG. 7B is a side view of the low-temperature manufacturing scaffold for use in tissue engineering according to the present invention, showing that the scaffold lacks any support structure; and

FIG. 8 is a flowchart of a manufacturing method for use in the low-temperature high-speed manufacturing of the support structure according to the present invention.

### DETAILED DESCRIPTION OF THE PREFERRED EMBODIMENTS

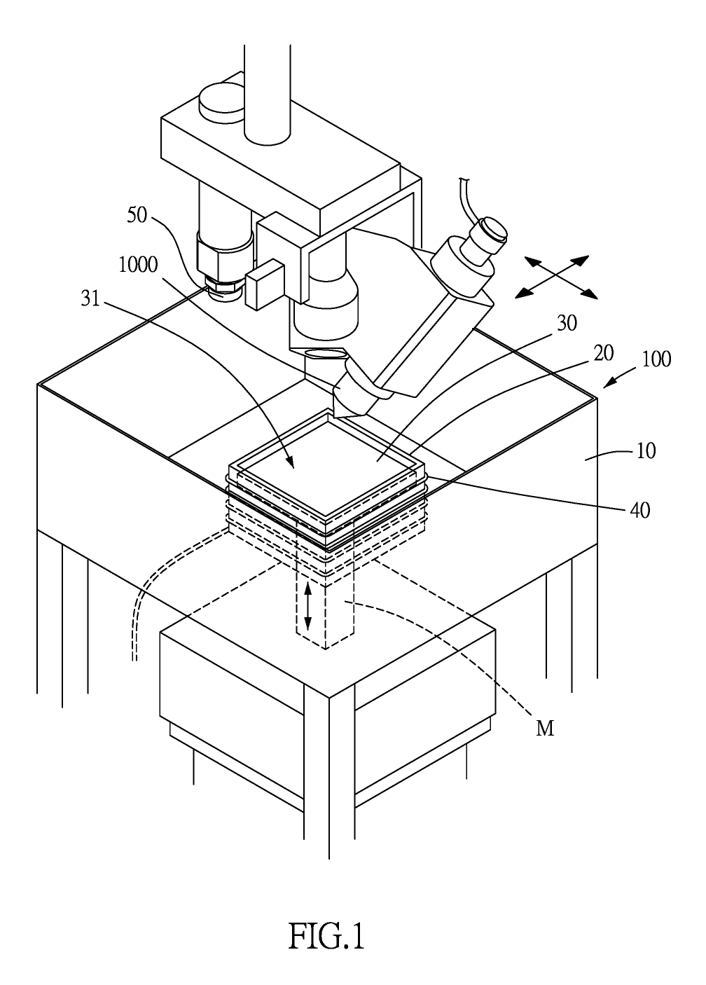

FIG. 1 shows a manufacturing apparatus 100 for use in the low-temperature high-speed manufacturing of a support structure. The manufacturing apparatus 100 and the support structure thus manufactured are for supporting a scaffold material layer of a low-temperature manufacturing scaffold for use in tissue engineering. The manufacturing apparatus 100 comprises a frame 10, a ring-shaped thermally conductive member 20, a thermally conductive platform 30, a low temperature generating mechanism 40 and a spray member 50. The manufacturing apparatus 100 comes with a nozzle 1000 to thereby manufacture a low-temperature manufacturing scaffold for use in tissue engineering. The nozzle 1000 injects a scaffold material layer (hereinafter referred to as the scaffold material layer) of the low-temperature manufacturing scaffold for use in tissue engineering such that the scaffold material layer deposits on the thermally conductive platform 30 of the manufacturing apparatus 100 or on the support structure manufactured with the manufacturing apparatus 100.

The frame 10 is disposed below the nozzle 1000.

The ring-shaped thermally conductive member 20 is fixedly disposed in the frame 10 and below the nozzle 1000. The ring-shaped thermally conductive member 20 is of a geometric shape, such as rectangular, round, or elliptical, but the present invention is not limited thereto. Preferably, the ring-shaped thermally conductive member 20 is made of stainless steel and thus resistant to corrosion and easy to sterilize.

The thermally conductive platform 30 is disposed centrally at the ring-shaped thermally conductive member 20 and has an edge in direct contact with the inner wall of the ring-shaped thermally conductive member 20. Hence, the ring-shaped thermally conductive member 20 and the thermally conductive platform 30 cool down each other. A space 31 is defined by and between the thermally conductive platform 30 and the ring-shaped thermally conductive member 20.

The low temperature generating mechanism 40 is connected to the ring-shaped thermally conductive member 20 to cool down the ring-shaped thermally conductive member 20. The low temperature generating mechanism 40 fits around the outer wall of the ring-shaped thermally conductive member 20. The low temperature generating mechanism 40 is a thermally conductive copper pipe that effectuates cooling by containing a coolant. Referring to FIG. 1, a peristaltic pump circulates a low-temperature alcohol solution within the low temperature generating mechanism 40 such that the low temperature generating mechanism 40 cools down the ring-shaped thermally conductive member 20, and in consequence the ring-shaped thermally conductive member 20 cools down the thermally conductive platform 30. In a variant embodiment of the present invention, the low temperature generating mechanism 40 is connected to the thermally conductive platform 30 to cool down the thermally conductive platform 30 and thus cool down the ring-shaped thermally conductive member 20. In another variant embodiment of the present invention, the low temperature generating mechanism 40 and the ring-shaped thermally conductive member 20 are integrated to form a single member. For example, channels are disposed inside the ring-shaped thermally conductive member 20 to convey a coolant to thereby form the low temperature generating mechanism 40, so as to enhance the cooling efficiency and reduce the required volume of the low temperature generating mechanism 40.

The spray member 50 is disposed above the thermally conductive platform to thereby spray a hydrated aerosol into the space 31. The hydrated aerosol (hereinafter referred to as the support material) is for use in manufacturing a support structure. Being hydrated, the support material facilitates low-temperature solidification. Being hydrated, the support material renders it easy to be removed from the low-temperature manufacturing scaffold for use in tissue engineering, even though the scaffold has a specific structure formed as a result of the deposition of the scaffold material layer on the support structure. With the spray member 50 spraying the hydrated aerosol extensively, the support structure can be manufactured quickly and distributed uniformly within the space 31. To enable the spray member 50 to spray the hydrated aerosol extensively, the nozzle of the spray member 50 is preferably fan-shaped, round or elliptical. In yet another variant embodiment of the present invention, to prevent the support material from remaining on the scaffold material layer, the spray member 50 sprays the hydrated aerosol with the airflow to thereby remove any residues of the support material from the scaffold material layer.

In a further embodiment of the present invention, the support material is pure water when its water content equals 100%, but the present invention is not limited thereto. Alternatively, the support material has a water content less than 100%, as is the case where the support material contains an alcohol solution, so as to adjust the freezing point.

Regarding the low-temperature manufacturing scaffold for use in tissue engineering and support structure, the low temperature must be low enough to allow the scaffold material layer and support material to freeze quickly. Hence, it is necessary that the low temperature generated from the low temperature generating mechanism 40 is changed according to the difference in the freezing point and the freeze duration between the scaffold material layer and the support material. The aforesaid cooling process is inevitably accompanied by a loss of cooling energy; hence, the low temperature generating mechanism 40 is always cooler than the ring-shaped thermally conductive member 20. For example, to allow the ring-shaped thermally conductive member 20, the thermally conductive platform 30 and the space 31 to reach −20□, the low temperature generated from the low temperature generating mechanism 40 must be lower than −20□.

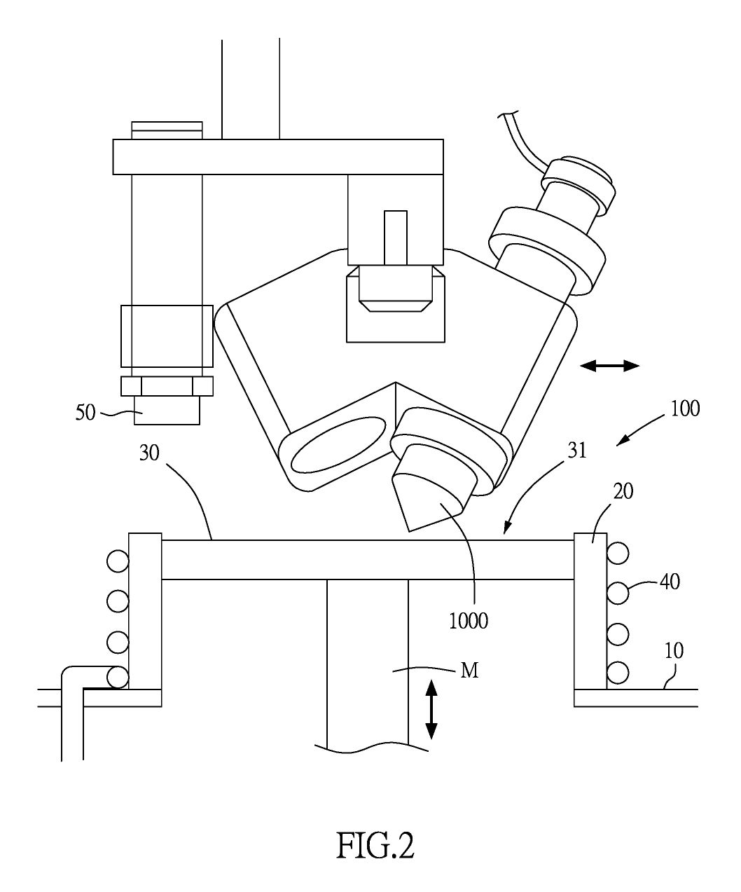

Referring to FIG. 1, the thermally conductive platform 30 is movably disposed at the ring-shaped thermally conductive member 20 and descends when driven by a vertically movable mechanism M, so as to increase the depth of the space 31 gradually and thus manufacture the low-temperature manufacturing scaffold for use in tissue engineering and the support structure at a constant temperature, as shown in FIG. 2 through FIG. 6. In a further variant embodiment of the present invention, the thermally conductive platform 30 is fixedly positioned at the ring-shaped thermally conductive member 20 to facilitate the manufacturing of the low-temperature manufacturing scaffold for use in tissue engineering and the support structure by moving the nozzle 1000 and the spray member 50 vertically.

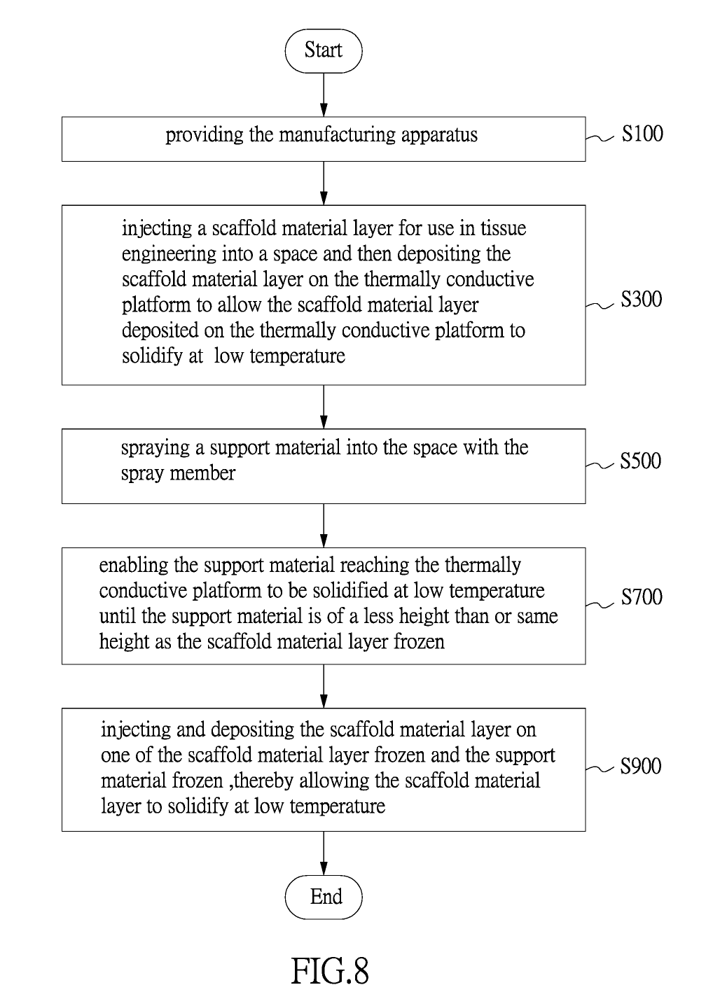

Referring to FIG. 2 through FIG. 8, both the manufacturing apparatus 100 and the nozzle 1000 enable the scaffold material layer to be deposited on the support structure at a low temperature such that the low-temperature manufacturing scaffold for use in tissue engineering forms a specific structure. FIG. 8 is a flowchart of a manufacturing method for use in the low-temperature high-speed manufacturing of the support structure according to the present invention.

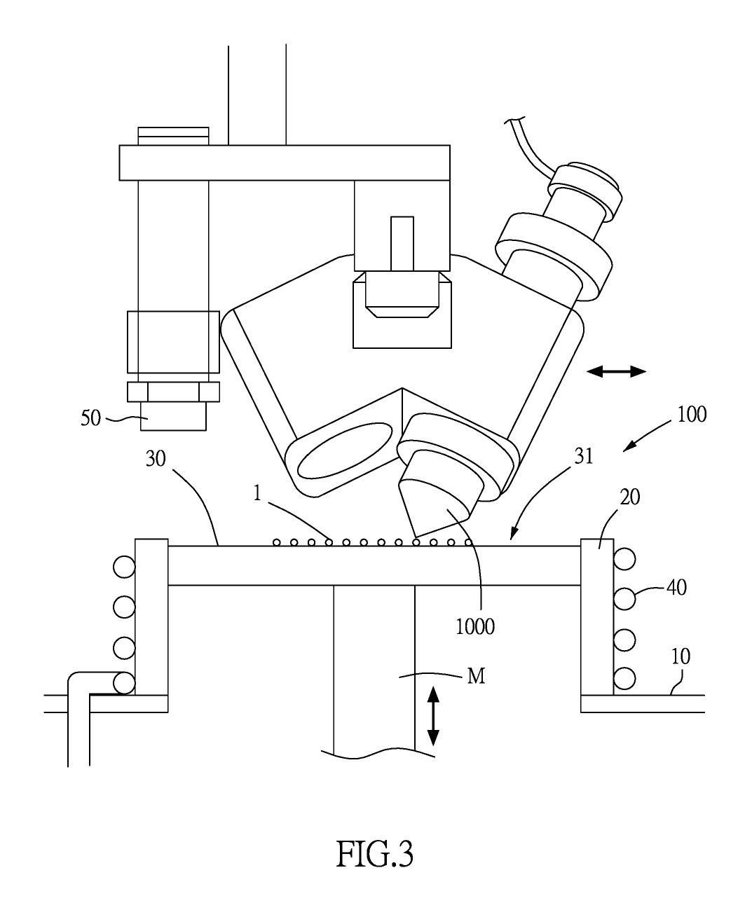

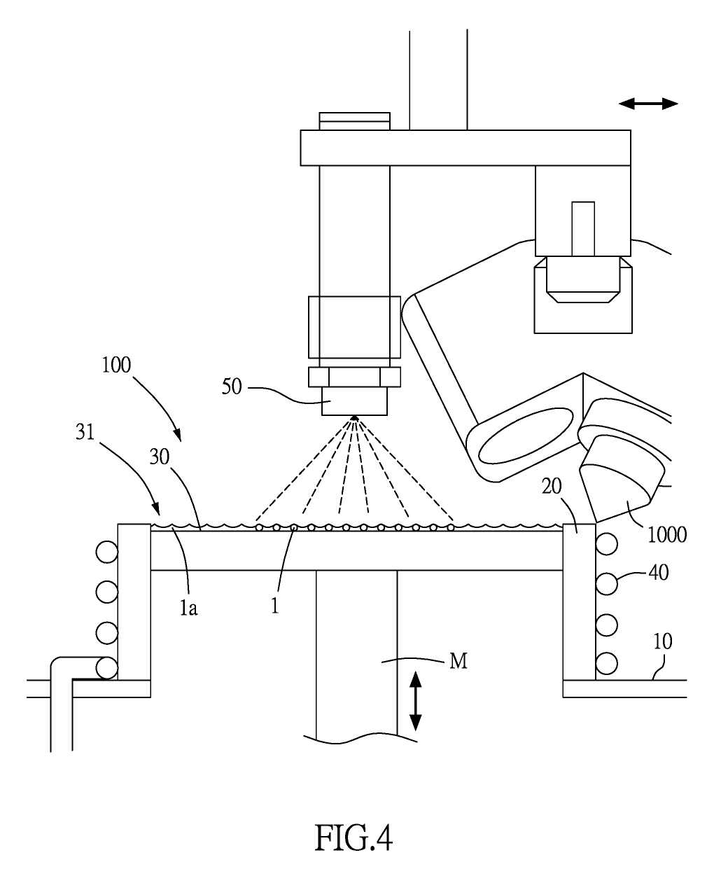

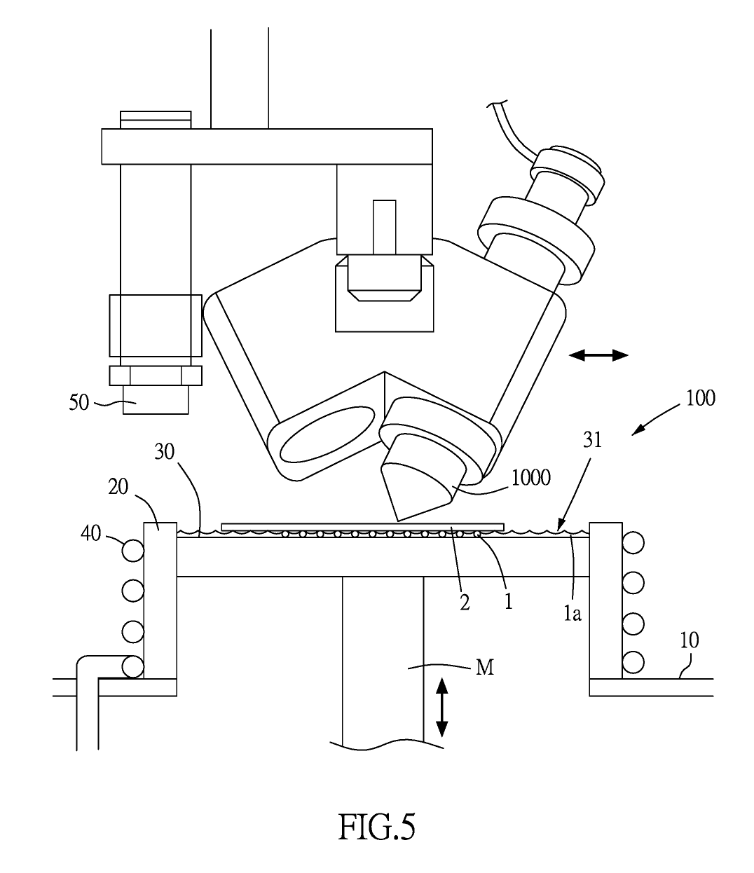

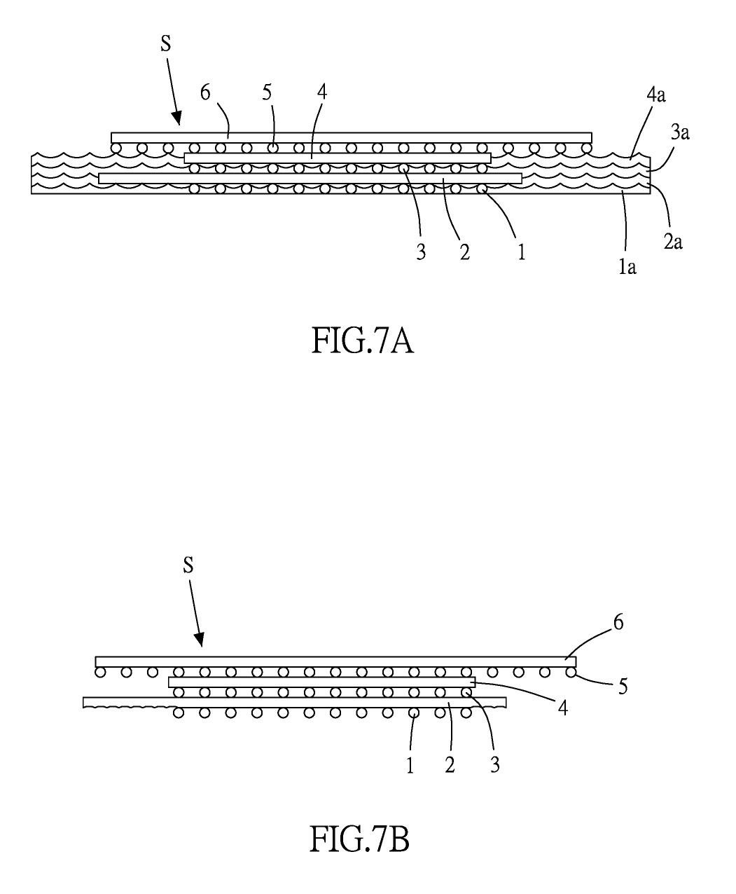

Referring to FIG. 2 and FIG. 8, in step S100, the manufacturing apparatus 100 is provided, and the thermally conductive platform 30 of the manufacturing apparatus 100 descends when driven by the vertically movable mechanism M, so as to define the space 31. Referring to FIG. 3 and FIG. 7, in step S300, in the space 31, the scaffold material layer is injected from the nozzle 1000, then deposited on the thermally conductive platform 30, and eventually solidified at a low temperature, so as to form a first scaffold material layer 1; to this end, the nozzle 1000 moves laterally relative to the platform structure 100, as indicated by the arrows shown in FIG. 1 and FIG. 2. Referring to FIG. 4 and FIG. 7, in step S500, the spray member 50 sprays the support material into the space 31, and the support material moves gradually to the thermally conductive platform 30. Referring to FIG. 4 and FIG. 7, in step S700, the support material freezes upon arrival at the thermally conductive platform 30, and the frozen process of the support material continues until the support material is of a less height than or same height as the scaffold material layer (i.e., the first scaffold material layer 1) frozen, thereby forming a first support material layer 1a. Referring to FIG. 5 and FIG. 7, in step S900, the scaffold material layer is injected from the nozzle 1000 once again, then deposited on the scaffold material layer frozen, (i.e., the first scaffold material layer 1) or the support material frozen, (i.e., the first support material layer 1a), thereby allowing the scaffold material layer to solidify, at low temperature and allowing a second scaffold material layer 2 to form; to this end, the nozzle 1000 moves laterally relative to the platform structure 100.

To allow the support material to be quickly and evenly distributed inside the space 31 and allow the height of the support material to increase quickly, the spray member 50 sprays extensively. To allow the support material to freeze quickly without being left on the scaffold material layer, it is necessary to control the temperature in the space 31 such that the support material efficiently flows to the thermally conductive platform 30 before getting frozen. To effectively prevent the support material from remaining on the scaffold material layer, the spray member 50 sprays the support material with the airflow simultaneously such that whatever residues of the support material is removed from the scaffold material layer.

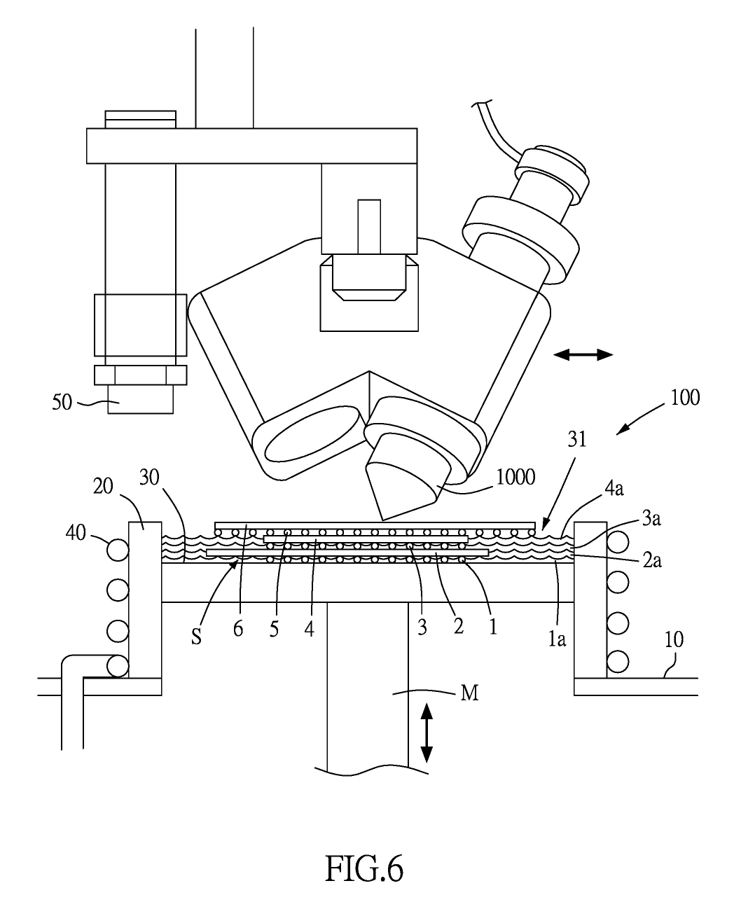

Step S500 through step S900 are carried out repeatedly to form a tissue engineering-oriented scaffold S of irregular shape as shown in FIG. 6. The tissue engineering-oriented scaffold S has six scaffold material layers and four support material layers. The second scaffold material layer 2 is deposited on both the first scaffold material layer 1 and the first support material layer 1a. A second support material layer 2a is sprayed on the first support material layer 1a. The second support material layer 2a is of the same height as the second scaffold material layer 2 substantially. A third scaffold material layer 3 is deposited on the second scaffold material layer 2. A third support material layer 3a is sprayed on both the second scaffold material layer 2 and the second support material layer 2a. The third support material layer 3a is of the same height as the third scaffold material layer 3 substantially. A fourth scaffold material layer 4 is deposited on the third scaffold material layer 3. A fourth support material layer 4a is sprayed on the third support material layer 3a. The fourth support material layer 4a is of the same height as the fourth scaffold material layer 4 substantially. A fifth scaffold material layer 5 is deposited on the fourth scaffold material layer 4 and the fourth support material layer 4a. A sixth scaffold material layer 6 is deposited on the fifth scaffold material layer 5.

In a further variant embodiment of the present invention, when the support material, namely the first support material layer 1a, the second support material layer 2a, the third support material layer 3a and the fourth support material layer 4a, freeze, their surfaces will be coarse so as to facilitate the deposition of the scaffold material layer thereon.

Referring to FIG. 7A, the tissue engineering-oriented scaffold S has a support material (the first support material layer 1a, the second support material layer 2a, the third support material layer 3a and the fourth support material layer 4a) which undergoes a freeze-drying process to effectuate the tissue engineering-oriented scaffold S having a scaffold material layer, as shown in FIG. 7B.

In conclusion, the present invention provides a manufacturing apparatus and a manufacturing method for use in the low-temperature high-speed manufacturing of a support structure to effectuate the low-temperature high-speed manufacturing of a support structure, facilitate the deposition of a scaffold material layer of a low-temperature manufacturing scaffold for use in tissue engineering on the support structure, and thus enable the low-temperature manufacturing scaffold for use in tissue engineering to form a specific structure.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

What is claimed is:

1. A manufacturing apparatus for use in low-temperature high-speed manufacturing of a support structure for supporting low-temperature manufacturing scaffold for use in tissue engineering, the manufacturing apparatus comprising:
a frame;
a ring-shaped thermally conductive member disposed on the frame;
a thermally conductive platform disposed centrally at the ring-shaped thermally conductive member and having an edge in direct contact with an inner wall of the ring-shaped thermally conductive member, wherein a space is defined by and between the thermally conductive platform and the ring-shaped thermally conductive member;
a low temperature generating mechanism connected to the ring-shaped thermally conductive member and the thermally conductive platform to cool down the ring-shaped thermally conductive member and the thermally conductive platform; and
a spray member disposed above the thermally conductive platform to spray a support material into the space.

2. The manufacturing apparatus of claim 1, wherein the support material has a water content of 100%.

3. The manufacturing apparatus of claim 2, wherein the spray member sprays the support material extensively.

4. The manufacturing apparatus of claim 3, wherein the spray member is fan-shaped, round or elliptical.

5. The manufacturing apparatus of claim 1, wherein the support material comprises an alcohol solution.

6. The manufacturing apparatus of claim 5, wherein the spray member sprays the support material extensively.

7. The manufacturing apparatus of claim 6, wherein the spray member is fan-shaped, round or elliptical.

8. The manufacturing apparatus of claim 1, wherein the spray member sprays the support material extensively.

9. The manufacturing apparatus of claim 8, wherein the spray member is fan-shaped, round or elliptical.

10. A manufacturing method for use in low-temperature high-speed manufacturing of a support structure, with the support structure supporting a low-temperature manufacturing scaffold for use in tissue engineering, the manufacturing method comprising the steps of:
S100: providing a manufacturing apparatus, the manufacturing apparatus comprises a frame; a ring-shaped thermally conductive member disposed on the frame; a thermally conductive platform disposed centrally at the ring-shaped thermally conductive member and having an edge in direct contact with an inner wall of the ring-shaped thermally conductive member, wherein a space is defined by and between the thermally conductive platform and the ring-shaped thermally conductive member; a low temperature generating mechanism connected to the ring-shaped thermally conductive member and the thermally conductive platform to cool down the ring-shaped thermally conductive member and the thermally conductive platform; and a spray member disposed above the thermally conductive platform to spray a support material into the space;
S300: injecting a scaffold material layer for use in tissue engineering into a space and then depositing the scaffold material layer on the thermally conductive platform to allow the scaffold material layer deposited on the thermally conductive platform to solidify at low temperature;
S500: spraying a support material into the space with the spray member;
S700: enabling the support material reaching the thermally conductive platform to be solidified at low temperature until the support material is of a less height than or same height as the scaffold material layer frozen; and
S900: injecting and depositing the scaffold material layer on one of the scaffold material layer frozen and the support material frozen, thereby allowing the scaffold material layer to solidify at low temperature.

11. The manufacturing method of claim 10, wherein the support material has a water content of 100%.

12. The manufacturing method of claim 11, wherein the spray member sprays the support material extensively.

13. The manufacturing method of claim 12, wherein the spray member is fan-shaped, round or elliptical.

14. The manufacturing method of claim 10, wherein the support material comprises an alcohol solution.

15. The manufacturing method of claim 14, wherein the spray member sprays the support material extensively.

16. The manufacturing method of claim 15, wherein the spray member is fan-shaped, round or elliptical.

17. The manufacturing method of claim 10, wherein the spray member sprays the support material extensively.

18. The manufacturing method of claim 17, wherein the spray member is fan-shaped, round or elliptical.
